(19) United States
(12) Patent Application Publication
(10) Pub. No.: US 2019/0159935 A1
(43) Pub. Date: May 30, 2019

(54) SURGICAL APPARATUS AND BLADE ELEMENTS FOR SLICING LAMELLAR SEGMENTS FROM BIOLOGICAL TISSUE

(71) Applicant: GEBAUER- KLOPOTEK PATENT VERWALTUNGS UG, Neuhausen (DE)

(72) Inventor: Peter J. Klopotek, Neuhausen (DE)

(73) Assignee: GEBAUER- KLOPOTEK PATENT VERWALTUNGS UG, Neuhausen (DE)

(21) Appl. No.: 16/323,629

(22) PCT Filed: Aug. 9, 2016
(86) PCT No.: PCT/IB2016/054793

§ 371 Date: Feb. 6, 2019

(51) Int. Cl. A61F 9/013

(52) U.S. Cl. CPC A61F 9/0133; A61B 2017/306

(57) ABSTRACT

Surgical apparatus and blade elements are provided for performing a tissue cutting process to cleave a tissue portion from a main tissue section. The apparatus comprises a reference member (1) having a cavity (3) for holding a tissue portion subject to the tissue cutting process and a blade guiding surface (2), a blade element (100, 200, 370, 470) adapted for slidable engagement with the reference member, such that the blade moves along the guiding surface of the reference member to perform the cutting process, and a biasing mechanism (80, 82, 91, 92, 102, 202) for moving the blade along the guide surface such that a pressure-relieving space is formed between the blade and the tissue section upon cleavage to avoid deviation of the cutting edge from a desired path. The blade elements can have a contact ridge (107) for guiding the blade element along the predetermined path. In some embodiments, the blade element can include a flexible plate member (101) having first and second opposite sides, a cutting edge (103) formed at a first longitudinal end portion of the plate member, and a manipulating block (102) mounted at a second longitudinal end portion of the plate member (101) for applying a biasing force to the plate member to maintain contact between the blade element and the reference member.

### FIELD OF THE INVENTION

The present invention relates to a surgical apparatus and blade elements for use in a surgical apparatus. In particular, the invention concerns a surgical apparatus for performing a tissue cutting process such as a removal of a biological tissue sample from a main section of biological tissue.

### BACKGROUND

Various techniques and devices are known in the prior art for performing a tissue cutting process, and in particular, for the removal of tissue using a blade element. These instruments are generally referred to as surgical microtomes. In specific applications, such surgical microtomes are designed for the removal of corneal tissue and in this case such instruments are referred to as keratomes or microkeratomes as well as microtomes.

The invention relates to a general surgical apparatus for performing a tissue cutting process and, for example, instruments for ophthalmologic applications to perform corneal surgery. In this specific application, the cutting or slicing process requires precision and accuracy due to the fact that one use of such a surgical apparatus can be a reshaping procedure of the cornea.

One class of microtomes, known as applanation microtomes, is illustrated by US Patent Application Pub. No. 2007/0265650 entitled “Device For Separating The Epithelial Layer From The Surface Of The Cornea Of An Eye.” In this instrument, a flat plate, known as an applanator, is used flatten the normal curvature of a cornea so that a blade can remove (or hingedly detach) a slice of corneal tissue of uniform thickness. (In some implementations, the applanator can take other shapes, e.g., like a “rolling pin,” and still serve to flatten the cornea directly in front of the associated blade.) The applanator will typically be drawn across the eye in front of the blade and the resected tissue will curl up into the space between the applanator and the blade in a manner similar to a wood shaving created by a carpenter's plane. There are several disadvantages to the use of such tissue-flattening structures. For example, applanation-microtomes cannot make refractive corrections (e.g., by removing a lenticule of non-uniform shape). Because of the geometry and operation of the applantor, the device is typically constrained to create a slice of uniform thickness.

Another approach to tissue cutting is disclosed in U.S. Pat. Nos. 6,030,398 6,099,541 and 6,626,924, all entitled “Surgical Microtomes.” In this approach, the tissue is not flattened but rather is held within a cavity of a reference member that presents a peripheral guiding edge for a cutting instrument. The shape of the cavity and/or the reference member's guiding edge can be varied to cut lamellar segments of any desired shape.

For example, when used for refractive corrections, an ocular reference member is employed to secure a portion of a cornea. The reference member is adapted to fixedly engage the cornea and has a peripheral, guiding edge for guiding a cutter (e.g., a blade), such that the cutter can be brought into physical contact with the guiding edge of the reference member and drawn through the cornea while maintaining physical contact with the guiding edge to detach a lamellar segment of the cornea of a desired shape. This class of microtomes are generally referred to herein as “rigid reference member” (“RRM”) or “peripheral reference member” microtomes.

However, a problem with RRM microtomes has been identified by the present inventor, namely that the cutting process can be affected by a build-up of pressure within the cavity of the reference member that forces the cutter away from firm contact with the guiding edge of the reference member. As a result, particularly in the final stages of a cut, deviations from an ideal resection are sometimes observed.

Accordingly, there exists a need for more accurate microtomes, particularly for removing tissue lamella of non-uniform shape, such as corneal lamella or lenticules for refractive correction.

### SUMMARY OF THE INVENTION

Methods and devices are disclosed for slicing lamellar segments, especially lenticules of non-uniform thickness from biological tissue, such as the cornea, with improved accuracy. The invention improves the accuracy of rigid reference member (RRM) microtomes by preventing hydrostatic pressure potentially exerted by the tissue enclosed in the cavity from causing the blade to disengage from the rigid reference member during the cutting process. This is achieved by a biasing mechanism that positions the blade in relation to the rigid reference member such that, at all times during the cutting process, a volume of space exists immediately behind the leading edge of the blade while a force is exerted about the leading edge of blade so that the blade maintains constant contact with the guiding edge of the rigid reference member that contains the tissue within its cavity.

In another aspect of the invention, blades are disclosed for use in a surgical apparatus by which the desired accuracy can be achieved with a simple and reliable construction. The present invention thus provides surgical apparatus and blades for performing a tissue slicing or cutting process with a predictable and highly reproducible result.

According to the first aspect of the present invention, a surgical apparatus is provided for performing a tissue cutting process to cleave a tissue portion from a main tissue section comprising:
a reference member having a cavity for holding a tissue portion subject to the tissue cutting process and a blade guiding surface,
a blade having a cutting edge for cutting the tissue portion to at least partially sever it from the main tissue section, the blade adapted for slidable engagement with the reference member, such that the blade moves along the guiding surface of the reference member to perform the cutting process, and
a biasing mechanism for moving the blade along the guide surface such that a pressure-relieving space is formed between the blade and the tissue section upon cleavage to avoid deviation of the cutting edge from a desired path.

In certain embodiments of the surgical apparatus, the blade can be biased at angle relative to the reference member by a bearing mechanism. In other embodiments, the biased by a bend or curve in the blade itself. Alternatively, the blade can be flexible such that the blade can be flexed by a bending moment. The flexibility of the blade can be achieved by providing the blade with a plate member made by a flexible plate shaped material, such as steel, or in particular spring steel.

In one preferred embodiment, the blade is biased by applying a bending moment. Various techniques can be employed to apply the desired torque as the blade is drawn along the guide element such that the blade remains engaged with the guide element during the resection procedure.

The surgical apparatus can also include a motor or other blade driver for driving the blade along the guiding surface in a cutting direction for driving the cutting edge between the tissue portion and the main tissue section is provided.

Such a motor/driver can apply a drive force to the blade at a designated portion thereof, e.g., at a opposite end of a blade shank from the cutting edge, in order to move the blade and, in particular, the cutting edge in the desired movement direction. This movement direction is defined as being aligned towards the cutting edge of the blade member such that the cutting edge is drivable into the tissue. The movement direction exerted by the driver is not strictly restricted to the direction along the guiding surface, as long as a deviation is within a predetermined limit.

Additionally, the driver can include an oscillator for transmitting an oscillating movement to the blade. The oscillating movement is directed in a substantially lateral direction in relation to the cutting direction. Based on this arrangement, the cutting edge can be oscillated by applying an oscillating movement to the blade at a designated portion thereof in order to optimize the cutting procedure. As such, the oscillating movement of the cutting edge and the movement for driving the blade along the guiding surface provide a combined movement of the cutting edge. The oscillating movement can be limited to a predetermined range in the substantially lateral direction of the blade.

The surgical apparatus can further include a cavity in the reference member. The cavity opens at the side where the guiding surface is provided. According to this arrangement, the cavity is arranged for accommodating a part of the tissue to be cut, in particular, the tissue portion which is to be cut or sliced away from the main tissue section. The cavity can be adapted in shape and size to the tissue portion as well as to the specific application. In some preferable embodiments, the cavity is arranged for accommodating and/or holding the tissue portion to be removed from the tissue main section in the cutting process.

According to this arrangement, the tissue portion to be removed from the tissue main section can be accommodated in the cavity, as discussed above, and can be held in the cavity in the cutting process. According to this arrangement, the tissue portion is held the cavity upon applying the cutting process. The tissue portion to be removed from the tissue main section can be held in the cavity after the cutting process is completed. In particular, the tissue portion cut away after completing the cutting process is held in a predetermined state, which includes the previous shape before initiating the cutting process.

The apparatus can further include one or more ports for applying a reduced (negative) pressure to the cavity of the reference member for holding the tissue portion to be removed in the cutting process. Reduced pressure can be applied at least in a part of the cavity in order to apply a suction effect to at least a part of a surface of the tissue portion to be removed. By such an action, the tissue portion to be removed from the main tissue section is securely held in place and/or maintained in a predetermined shape. In certain embodiments, at least a portion defining the cavity is formed as a permeable structure for applying reduced pressure to the cavity.

With such an arrangement, the structural integrity of the surface defining the cavity can be maintained while at the same time a reduced pressure can be applied to at least a part of the surface of the tissue portion to be removed in the cutting process. The above mentioned air permeable structure can be in fluid connection to the reduced pressure port for applying reduced pressure which can on the other hand be connected to a low pressure source for applying the required low pressure to the cavity. As alternative or in addition, the tissue portion can be held in the cavity by adhesion, e.g. by the use of adhesive.

Additionally, the surgical apparatus of the invention can comprise the following:
a reference member having an arrangement for holding the tissue portion subject to the tissue cutting process and a guiding surface, and
a blade for cutting the tissue portion subject to the cutting process, the blade having a contact ridge which is slidably engageable to the guiding surface of the reference member.

During the cutting process, the blade is moved along the reference member. In a preferred embodiment, the blade is provided with a contact ridge which is adapted to be in contact with the reference member and the predetermined position of the blade with respect to the reference member is achieved by a contact of the contact ridge and the surface of the reference member. Upon performing the cutting process, the blade is moved along the surface of the reference member, in particular with the contact ridge contacting the guiding surface of the reference member.

The contact ridge of the blade can be kept in contact to the surface of the reference member by a force, e.g., a counterforce resulting from a biasing mechanism. The operation of guiding the blade is achieved by keeping a close contact between the contact ridge and the surface of the reference member upon moving that blade. Thus, the contact ridge slides along the surface of that reference member with reduced friction. A second guiding surface on the other side of the blade, that is the side opposite to the side where the contact ridge is provided, is not required as long as the force created by biasing mechanism is sufficient to maintain close contact between the contact ridge and the surface of the reference member.

The guiding mechanism of the reference member includes a guiding surface. The surface can also be an edge of the reference member. The terms “guide,” “guiding surface” and “guiding edge” are used interchangeably throughout this description unless it is otherwise necessary to distinguish between an essentially two-dimensional and an essentially one-dimensional structure. Preferably, the contact ridge of the blade is in pressure contact with the guiding surface at least during the tissue cutting process. The use of the guiding surface provides a specific degree of freedom for designing the movement path of the blade insofar as the guiding surface need not be planar. In particular, the cutting path of the cutting edge provided at the blade. Moreover, the above-mentioned pressure contact establishes the required guiding function as long as this pressure contact ensures that a departure of the contact ridge from the guiding surface of the reference member is avoided.

As noted above, the blade can be mounted in the surgical apparatus by a biasing mechanism in an angular orientation, or in a curved or flexed state such that the contact ridge is forced against the guiding surface for defining the movement path in the cutting process. Alternatively, the reference member can be curved (cylindrically concave) in shape to provide the pressure relief.

The blade can be usable for a number of operations or surgery actions and can be replaceable by a new one when necessary e.g. when tissue abrasion has reduced the sharpness of the cutting edge. Alternatively, the blade can be intended for a single use only and replaced after the completion of a single cutting operation. However, reusing of the blade is not excluded.

The blade is preferably shaped such that a designated element, e.g., a contact ridge, is essentially the sole contact with the guiding surface, such other elements, in particular the cutting edge, can be spaced apart from any element which is in contact with the guiding surface during the cutting process. By such an arrangement, the operation of the cutting edge is not impaired as the cutting edge can freely and independently move along the desired movement path. The specific arrangement of the blade and the surgical apparatus can establish a space between surfaces of the blade, which face towards the guiding surface provided in the surgical apparatus. In particular, the contact between the blade and the guide in the surgical apparatus can be established only by the contact ridge while specifically those areas of the blade which are located opposite to the cutting edge with respect to the contact ridge are spaced from the surface or surfaces of the guiding surface. Friction and interference between the cut surface of the tissue to be cut or sliced and the blade can be largely avoided, which increases the accuracy and the reliability of the blade in operation to a high extent. The contact ridge can be linear or curved. The contact ridge can be unitary in structure or can be formed by a plurality of discontinuous ridge segments.

Additionally, the surface of the blade facing to the guiding surface can be inclined with respect to the guide in the cutting process such that a space is formed between the blade and a cut surface of the tissue portion to be removed.

The specific arrangement of the blade (relative to the guide surface of the reference member) provides a space behind the area in which the cutting operation is taking place such that engagement of the severed tissue within the cavity and surface elements of the blade is avoided as much as possible. This improves the cutting procedure as the friction force between the blade and the resected tissue is reduced. Therefore, the position of the tissue portion in the cavity can be maintained unchanged while a possible excess of the tissue portion can be accommodated in the space, thus avoiding any disturbance of the resected tissue portion held in the cavity. The space also provides relief for hydrostatic pressure that can otherwise build-up in the cavity during the cutting process. (The term “hydrostatic” is used to approximate the behavior of tissue, especially ocular tissue, which is not actually a fluid but rather a viscoelastic material that exerts a hydrostatic-like pressure when partially enclosed.)

In certain embodiments, a fixing arrangement, such as suction mechanism, is also provided for at least partially fixing the main tissue section in predetermined relationship to the reference member. For example, in the context of ocular surgery, the fixing arrangement can be a suction ring that surrounds the sclera of the eye and fixedly holds the eye so that the apparatus can cleave or partially detach a slice of the cornea.

This fixing arrangement is provided for enabling a defined positional relationship between the main tissue section and the tissue portion to be removed, which enhances the accuracy of the cutting process. The above-mentioned relationship between the main tissue section and the reference member can be achieved by a substantially fixed relationship between the fixing arrangement and the reference member in one example. As alternative, the above-mentioned predetermined relationship can be achieved by an arrangement for controlling or adjusting the positional relationship between the fixing arrangement and the reference member.

In certain embodiments, the fixing arrangement comprises a fixing recess for applying a lower pressure to a portion of the main tissue section for fixing the main tissue section in relation to the fixing arrangement and/or the reference member. Upon applying the low pressure, a part of the main tissue section can be sucked partially into the fixing recess and brought into contact to a surface inside the fixing recess. The fixing recess can, in a further embodiment, include an air permeable structure in order to apply the required low pressure to at least a part of a surface inside the fixing recess in order to apply a force for holding the main tissue section. It is possible to provide multiple fixing recesses.

According to a preferable embodiment, the main tissue section is an eye and the tissue portion to be removed in the cutting process is a portion of a cornea of the eye. As such, in this preferable embodiment, the surgical apparatus is provided for holding a main tissue section of an eye in the fixing recess, whereas a portion of the cornea of the eye is held by the cavity provided in the reference member. In this instance, the fixing recess is provided as annular recess or depression arranged for holding an annular part of the eye which is located in an area of the eye surrounding the cornea subject to the cutting section. For example, the main tissue section can be the scleral ocular globe and it can be held in place by a suction ring while the cornea is engaged by the rigid reference member.

It is noted, that a complete removal of the tissue portion from the main tissue section is not strictly required in the operation of the apparatus. Rather, a partial cutting process is covered by the above-mentioned expression of cutting. Moreover, the cutting process can also be understood as a slicing, cleaving or shaving process due to the fact that tissue portion to be removed from the main tissue section is a very thin tissue element in the range of few micrometers in some exemplary applications.

Although in the general description of the invention, the application is partially related to a surgical apparatus applicable to an eye, any cutting process applied to tissue is covered by the above explanation. In particular, the above-mentioned surgical apparatus can be understood as microtome indicated above with reference to various known techniques and devices. However, based on the above inventive concept, the accuracy of the cutting operation based on a blade and the corresponding surgical apparatus is enhanced while the arrangement for providing the accuracy in particular for moving the cutting edge along a predetermined path is simplified.

According to another aspect of the present invention, a blade for use in surgical apparatus having a guiding surface for guiding the blade along a predetermined path is provided. The blade comprises the following:
a plate member having first and second opposite sides,
a cutting edge formed at a first longitudinal end portion of the plate member, and
a contact ridge formed at the first side of the plate member at a predetermined distance from the cutting edge with respect to a longitudinal direction of the plate member, the contact ridge being arranged for slidably engaging the guiding surface formed in the surgical apparatus when blade is driven in a cutting action.

The blades of the present invention can further include a biasing mechanism or can be adapted to cooperate with a biasing mechanism to ensure a proper “attack” angle for the blade's cutting edge in use. For example, the blade can include (or have an element that cooperates with) a bearing, a curved surface or a biasing force generator.

In certain embodiments, the blade can be flexible so that it can be flexed relative to the blade's longitudinal axis by application of torque or a bending moment to bias the cutting edge of the blade is a desired angular orientation relative to the guiding edge and/or the intended cleavage path. For example, the blade can be sufficiently thin and capable of flexure relative to the longitudinal axis such that a manipulating member mounted at a second longitudinal end portion of the plate member (the end opposite the cutting edge) can apply at least a bending moment to the plate member. The terms “manipulator,” “manipulating member” and “manipulating block” are used interchangeably herein to describe a handle or stock element that joins the blade to the RRM and engages directly or indirectly with a biasing element to apply a force to blade that counteracts any tendency of the blade to deviate from a desired cutting path. In certain embodiments, described in more detail below, the manipulating block can also have an irregular shape, e.g., a polygonal shape, to fit into a locking keyhole, to prevent dislodgement of the blade or cutter from the RRM during use. In use, the applied torque at a distal end of the blade can be between 10 and 200 grams or more preferably in some instances between 30 and 70 grams.

The blade can be used in a surgical apparatus for performing a tissue cutting process. For performing the cutting process, the blade is provided with the cutting edge at one end portion, which can be moved into the tissue to be cut or sliced. The surgical apparatus further includes a biasing mechanism applies a force and/or a bending moment to the shank plate of the blade. In certain embodiments, the blade is designed to bow, e.g., convexly, in response to the torque application by the manipulating block. The blade is preferably made of metal, and more preferably comprises a high performance steel, such as Sandvik 13C26 chromium steel (available from Sanvik Materials Technology, Storgatan, Sweden) or Hitachi GIN5 stainless steel (available from Hitachi Metals Ltd. of Nagoya, Japan)

The plate member of the blade can formed as flexible plate member such that a bent or flexed state of the plate member is achievable. In certain preferred embodiments, the flexible plate member is mounted to the manipulating block and a bending moment can be applied to the plate member in order to bend or flex the plate member. For example, the blade bending angle can be between 0.3 and 3 degrees, or between 0.3 and 2 degrees, or between 0.5 and 1 degree. The precondition for the flexed state of the plate member is a counterforce, which is acting at a section of the flexible plate member, which is remote from the manipulating member of the blade.

The contact ridge of the blade can be formed at a segment of the blade where a counterforce can be created due to a flexed state of the flexible plate member. The contact ridge is arranged on one side of the plate member. The contact ridge is arranged such that a sliding movement of the contact ridge along the guiding surface is enabled. This sliding movement can be achievable by moving or actuating a manipulating member of the blade. The predetermined distance between the contact ridge and the cutting edge is such that the sliding movement of the contact ridge governs the movement path of the cutting edge. In particular, the distance between the cutting edge and the contact ridge is short enough for limiting a substantial deviation of the relative positions of the cutting edge and the contact ridge.

The contact ridge can be adapted for a sliding movement in a lateral direction of the plate member, i.e. in a direction which is approximately aligned to the cutting edge or which is substantially perpendicular with respect to the movement direction into the tissue to be cut or sliced.

The above-mentioned guiding surface can be formed in a surgical apparatus and adapted in properties to the shape of the blade and/or to properties of the contact ridge. The reference member can include a guiding surface along which the contact ridge can be slidably moved while the contact ridge is engaged to the surface by a force which is created by the above mentioned bending moment, which can be applied to the plate member at the manipulating member. The contact ridge can be arranged for an engagement with multiple surfaces optionally provided in the guide. Again, the contact ridge can be unitary in structure or can be formed by a plurality of discontinuous ridge segments.

According to a preferable embodiment, the plate member comprises a main section and a tip section, wherein the main section has a first main surface at the first side and a second main surface at the second side, the tip section extending from the main section and includes the cutting edge.

According to a preferable embodiment, the main section is formed with a substantially constant thickness between the first main surface and the second main surface. However, it is not excluded that sections with deviating thickness ranges are provided, such as mounting elements, holes, windows or depressions as long as the major part of the main section is formed with a substantially constant thickness as stated above.

The tip section includes the contact ridge and the cutting edge, which are arranged in a predetermined relationship. The tip section has a varying thickness along the longitudinal direction of the blade. The thickness of the remaining portion of the blade, i.e. the main section is provided with a substantially constant thickness as stated above. The main section of the plate member can be provided with a constant width. Due to such an arrangement, the production of the blade is simplified and, at the same time, the properties of the plate member with respect to the bending moment in view of the flexibility and the counterforce to be generated at the contact ridge are easily reproducible.

According to a preferable embodiment, the tip section is formed by a first taper surface on the first side of the plate member and a second taper surface at the second side of the plate member. The first and second taper surfaces merge into the cutting edge. The lengths of the first and second taper surfaces are preferably different with respect to the longitudinal direction of the plate member. The taper angle in relation to the longitudinal direction of the plate member is preferably different for the first taper surface and the second taper surface. The tip section can be defined by a front tip section including the first taper section and a part of the second taper section. A rear tip section can defined between the contact ridge and the main section of the blade.

According to a preferable embodiment, a position of the cutting edge is offset towards the first side of the plate member with respect to a symmetry plane defined in the main section between the first and second main surfaces.

According to this arrangement, the accuracy of positioning the cutting edge can be improved in the intended operation due to the fact that the cutting edge is offset in the thickness direction of the main section towards the surface of the plate member at which the contact ridge is arranged. In particular, the cutting edge is offset from a neutral axis of the plate member such that a deviation of the positional relationship of the cutting edge and the guiding surface e.g. upon variations of the flexing state of the plate member can be minimized.

According to a preferable embodiment, a distance of an offset plane which is oriented parallel to the first main surface of the main section and which includes the cutting edge is less than 50 percent, preferably less than 40 percent of the thickness of the main section.

Based on the above arrangements, the accuracy of positioning the cutting edge can be improved while the manufacturing process of the blade is maintained as simple as possible. Moreover, based on such an arrangement, a predetermined distance between the cutting edge and the guiding surface in a longitudinal direction of the blade can be assured in order to protect the cutting edge from abrasion by the RRM during movement.

In certain embodiments, the contact ridge is formed at a transition of the first taper surface and the first main surface. According to this arrangement, the contact ridge can be formed by the transition of the first taper surface and the main surface by the resulting inclined arrangement between the surfaces. Therefore, inclination of the first taper surface has the function of decreasing the thickness of the plate member in order to form the cutting edge and, at the same time, achieves the provision of the contact ridge at the transition of both surfaces. As such, the contact ridge can be formed by the border or edge where the surfaces meet. This can preferably be achieved without adding material. The contact ridge can be smoothened by at least partially machining the border or edge between the surfaces or can be maintained as sharp as it is.

According to one embodiment, the contact ridge protrudes from the first taper surface and the first main surface. Additional material can be provided at the transition of the first taper surface and the first main surface. Such a form for the contact ridge can be designed with a desirable shape such as a circular or elliptical shape in a cross section. Moreover, the contact ridge can extend in the lateral direction substantially throughout the entire width of the plate member. As alternative, the contact ridge can be limited to a specific width range of the plate member. Moreover, it is possible to provide multiple contact ridge sections that are interrupted along the lateral direction of the plate member.

According to another embodiment, a distance between the contact ridge and the cutting edge in the longitudinal direction of the main section is 5-150 micrometers preferably 20-70 micrometers. Based on such a relationship, the accuracy for guiding the cutting edge is optimized while the manufacturing process of such a plate member is simplified. In particular, the above mentioned distance is short enough in order to secure the accuracy of guidance of the cutting edge while the above mentioned distance is long enough for enabling the manufacture of the plate member with a reasonable effort. In particular, a sufficient distance between the cutting edge and the contact ridge is required for enabling an optimum cutting property of the cutting edge.

According to yet another embodiment, a thickness of the main blade section is 20-500 micrometers, preferably 150-350 micrometers. A blade having the above-mentioned properties provides the required flexibility properties while the mechanical stability of such a blade is secured. The invention encompasses blades that have all of the above-recited dimensional characteristics or any combination thereof.

The blades of the present invention can be applied in a surgical apparatus that comprises a blade guide having one or multiple surfaces serving as guide for the contact ridge provided in the blade according to the first aspect. The specific arrangement of the blade can establish a space between surfaces of the blade that face towards the guiding surface provided in the surgical apparatus. In particular, the contact between the blade and the guiding surface in the surgical apparatus is established only by the contact ridge while specifically those areas of the blade which are located opposite to the cutting edge with respect to the contact ridge are spaced from the surface or surfaces of the guide. Friction and interference between the cut surface of the tissue to be cut or sliced and the blade can be avoided as much as possible which increases the accuracy and the reliability of the blade in operation to a high extent.

The invention is explained in detail based on the following drawings, which show examples of the blade and the surgical apparatus in which such a blade is usable. It is noted that the invention is not restricted to the below detailed explanation which serves as clarification of the basic concept of the invention only which is defined in the claims.

### BRIEF DESCRIPTION OF THE DRAWINGS

FIG. 1 is a schematic perspective view of a prior art “rigid reference member” microtome.

FIG. 1A is a schematic cross-sectional view of an initial stage of a tissue-cutting procedure with a prior art microtome such as shown in FIG. 1.

FIG. 1B is a schematic cross-sectional view of a subsequent stage of the operation of the prior art microtome of FIG. 1A, illustrating the potential drift of the blade from the desired path due to pressure build-up in the cavity of the reference member.

FIG. 2 is a schematic illustration of one embodiment of a surgical apparatus according to the invention in which a bearing applies a biasing force to maintain at an angle relative to cutting path to provide a pressure-relief space behind the leading edge of the blade as it cleaves the target tissue.

FIG. 3 is a schematic illustration of another embodiment of a surgical apparatus according to the invention in which the blade has a curved segment to provide the desired pressure-relief space behind the leading edge of the blade.

FIG. 4 is a schematic illustration of yet another embodiment of a surgical apparatus according to the invention in which a bending torque is applied to a flexible blade such that the cutting edge of the blade is disposed at an angle relative to cutting path to provide the desired pressure-relief space behind the leading edge of the blade.

FIG. 5 is a schematic illustration showing portions of the blade in greater detail including a contact ridge on the blade;

FIG. 5A is an enlarge view of the contact ridge of FIG. 5;

FIG. 5B is a schematic perspective view of the blade of FIGS. 4 and 5;

FIG. 6 is a schematic illustration showing portions of the blade in more detail;

FIG. 7 is a schematic perspective view of another embodiment of a blade according to the present invention having a central cut-out;

FIG. 7A is a schematic top plane view of the blade of FIG. 7;

FIG. 7B is a schematic perspective view of the blade of FIGS. 7 and 7A;

FIG. 8A is a schematic side view of another embodiment of the present invention in which the reference member is curved to relieve pressure build-up in the reference member's cavity;

FIG. 8B is schematic perspective view of the curved reference member embodiment of FIG. 8B;

FIG. 9 is a schematic, partially cut-away, side view of an ocular surgery apparatus according to the invention having a stationary guide element for holding an eye and a translatory blade and driver element.

FIG. 10 is a more detailed schematic side view of the apparatus of FIG. 9 also showing an associated eye-fixation suction ring;

FIG. 11 is a partially cut-away, side view of the apparatus of FIGS. 9 and 10 with the suction ring and drive mechanism removed;

FIG. 11A is a schematic cross-sectional, side view showing one mechanism for inserting a blade element and mounting block into an apparatus according to the invention;

FIG. 11B is a schematic cross-sectional, side view showing another mechanism for inserting a blade element and mounting block into an apparatus according to the invention;

FIG. 12 is another partially cut-away, side view of the apparatus of FIGS. 9 and 10 showing a mechanism for imparting oscillatory motion to the blade of the apparatus;

FIG. 13 is a schematic, partially cut-away, perspective view of the apparatus of FIGS. 9-12 showing the blade and block assembly inserted into a mating receptor or keyhole within a driver element of the apparatus;

FIG. 14 is a cut-away, end view of the stationary element of the apparatus of FIGS. 9-13 showing rails for engaging the translatory blade and drive elements of the apparatus;

FIG. 15A is a schematic, partially cut-away, side view of an eye fixation ring according to one embodiment of the invention; and

FIG. 15B is a schematic side view of another embodiment of the invention showing a tissue fixation stage in lieu of an eye fixation device.

### DETAILED DESCRIPTION

In the following, embodiments of the present invention are explained based on the drawings. The surgical apparatus will be explained first followed by an explanation of the blade element. It is noted that the present invention concerns the surgical apparatus having the blade element as well as the blade element as independent subject matter. The terms “blade” and “blade element” are used interchangeably and generally throughout this description to describe any mechanism capable of cutting, slicing or cleaving.

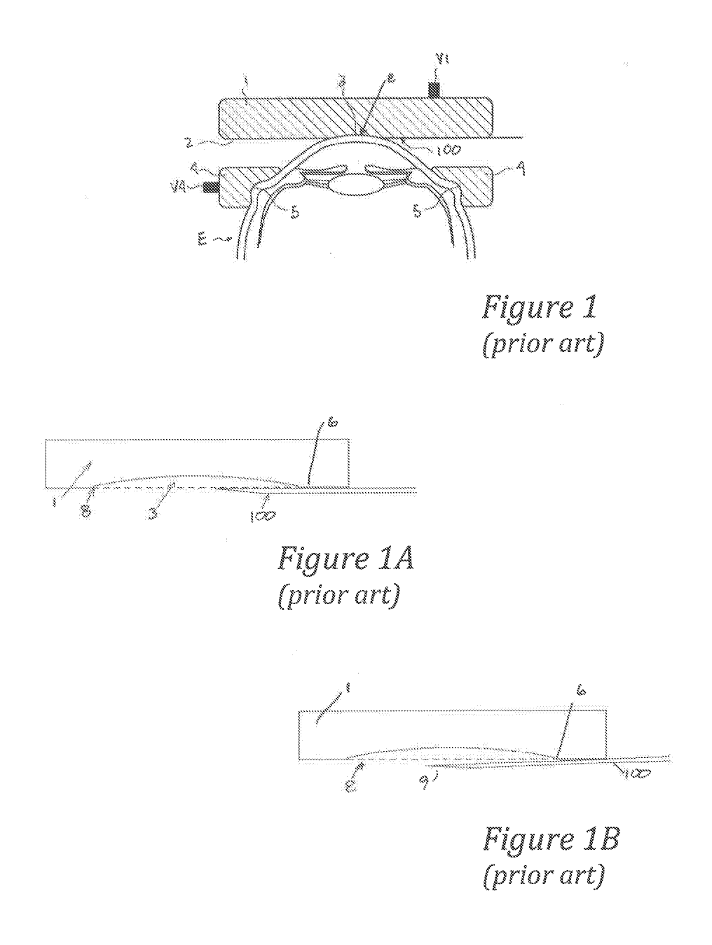

However, a general description of a prior art reference member microtome is first warranted. FIG. 1 shows such a surgical apparatus in an exemplary embodiment, which can be applied to a surgery in an eye. In FIG. 1, the eye is shown as example of a main tissue section E from which a part of the cornea as tissue portion e is to be cut or sliced. The surgical apparatus includes a reference member 1 which is shown with a simplified block shape. In the reference member 1, an arrangement for holding the tissue portion e subject to the tissue cutting process is provided. In the present embodiment, the arrangement for holding the tissue portion e is formed as cavity 3. The cavity is formed in the reference member 1 in the side facing towards the main tissue section E. In the prior art embodiment, a part of the tissue main section E, namely the tissue portion e to be removed from the main tissue section E is placed in the cavity 3.

In order to make sure that the tissue portion e is held in position, the arrangement for holding the tissue portion e can include a system for applying a reduced pressure to the cavity 3. This system for applying a reduced pressure to the cavity 3 can be formed by a porous material in the area of the cavity, which is permeable for air. This permeable structure can be fluidly connected to a vacuum port V1 to which a reduced pressure can be applied. It is noted that the use of porous material is not strictly required as long as a low pressure can act at the tissue portion e for holding the same in place. In particular, it is possible to provide one or multiple openings in the surface defining the cavity 3 which are in fluid connection with the vacuum port V1. As alternative or in addition to the system using the reduced pressure, adhesive can be used to hold the tissue portion e in the desired location. Although the apparatus and methods of the present invention are generally illustrated for use in refractive correction of a live eye, it should be clear that the invention can also be used on other tissue. Additionally, the invention can used to remove lamella from donor eyes or donor cornea. In such applications the tissue suction ring would simply be replaced by appropriate staging to ensure that the tissue is presented to the blade in a fixed position.

The reference member 1 includes a guiding surface 2 that is formed on the side of the reference member 1 facing towards the main tissue section E. In the present embodiment, the guiding surface 2 is formed as surface in the reference member, which surface provides a guiding function for the blade element 100. The surface forming the guiding surface 2 can be provided in the peripheral area of the cavity 3. The surface of the guide 2 can be formed as straight surface or alternatively the surface can be designed with a specific curvature or predetermined shape in order to provide a desired guiding path for guiding the blade element 100.

It is also possible to provide multiple surfaces forming the guide 2 which are for example arranged laterally on opposing sides of the reference member 1 with the cavity been located in between. In any case, the one or multiple surfaces forming the guiding surface are provided in order to extend along a path in which the blade element 7 is to be guided upon performing the cutting process explained below.

As additional measure for securing the position of the main tissue section E as well as the tissue portion e, a fixing arrangement 4 for at least partially fixing the main tissue section E can e.g. in the form of a suction ring be proved. In the present embodiment, with the application of the surgical apparatus to a surgery in an eye, the fixing arrangement 4 comprises a member in which a fixing recess 5 is provided. In the present example, the fixing recess 5 is formed as annular depression in the radial inner surface of the member having the fixing recess 5. A reduced pressure can be applied to the fixing recess 5 in a similar manner as explained in relation to the cavity 3 above. In particular, a porous arrangement can be provided in the fixing arrangement 4 that is in fluid connection to a vacuum port V4. In another implementation the fixing arrangement 4 can be design as a so-called vacuum ring made of a dense material (e.g., metal or plastic, having a vacuum port directly connected to tissue. When the tissue is corneal tissue, the viscoelastic properties of the eye and the intraocular pressure can be employed to ensure fixation when negative pressure is applied to the ring. When applying the surgical apparatus to a surgery in an eye, a portion of the main tissue section E can be held by applying a reduced pressure in the fixing recess 5 while the portion of the main tissue section E to be held in position is arranged in contact to the fixing arrangement 4. The vacuum port V4 transmits the reduced pressure to the portion of the main tissue section E such that a deformation can takes place and this portion of tissue is held at the fixing arrangement 4.

The fixing arrangement 4 can be arranged with a constant relationship to the reference member 1. As alternative, the positional relationship between the fixing arrangement 4 and the reference member 1 can be adapted to the specific application. In this case, a specific arrangement can be provided for controlling the positional relationship between the reference member 1 and the fixing arrangement 4. This arrangement can include a manually operated arrangement or can make use of actuators that can be controlled by a system control optionally provided.

FIGS. 1A and 1B illustrate the problem sometimes encountered with prior art rigid reference member (RRM) microtomes, namely that the cutting process can be affected by a build-up of pressure within the cavity 3 of the reference member 1 as the blade 7 is drawn along the desired path 8 defined by the guiding surface 6 of the reference member 1.

As shown in FIG. 1B, build-up pressure (hydrostatic pressure) forces the blade 7 away from firm contact with the guiding surface 6 of the reference members. As a result, particularly in the final stages of a cut, a deviation 9 from an ideal resection is sometimes observed.

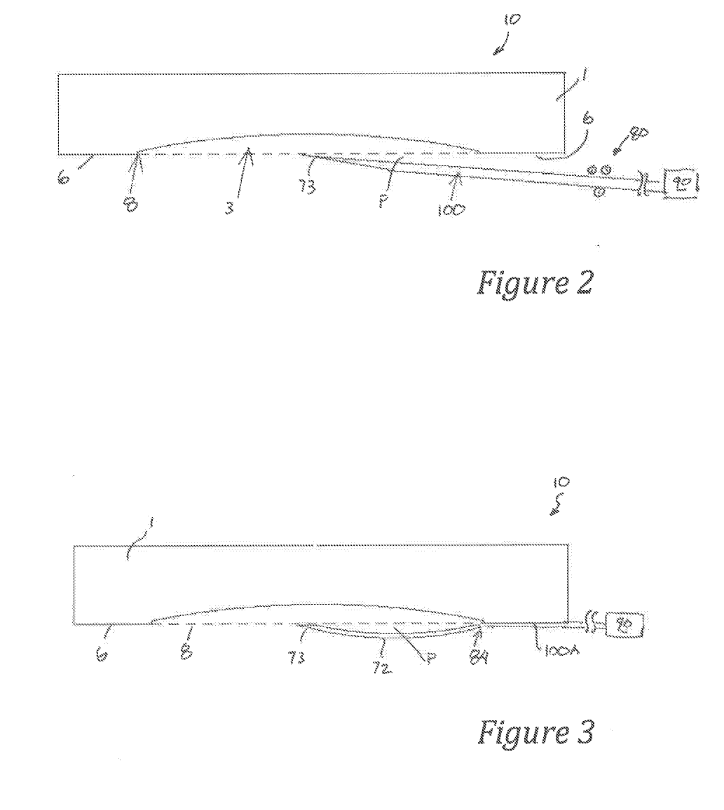

FIG. 2 illustrates one embodiment of a surgical apparatus 10 of the present invention, in which the reference member 1 presents a guiding surface 6 like that shown and described in FIG. 1—but the blade 70 is biased at angle relative to the guiding surface 6 of the reference member by a bearing mechanism 80. As the blade 70 is driven along the guiding surface 6 by motor 90, the cutting edge 73 is forced into contact with the guiding surface 6 so that blade will slice tissue within cavity 3 along the desired path 8 and not deviate from path 8 due to hydrostatic pressure build-up within the cavity or any other causes. The angle between the blade 70 behind the cutting edge 73 forms a space P that permits expansion of the severed tissue and consequent relief of pressure build-up.

In FIG. 3 another embodiment of a surgical apparatus 10 of the invention is shown, in which a reference member 1 again presents a guiding surface 6 like that shown and described in FIGS. 1 and 2—but the blade 70A is the biased by a bend or curve 72 in the blade itself between the cutting edge 73 and shank 84 of the blade 70A. The bend 72 in the blade 70A provides a pressure relief space P that accommodates expansion of the resected tissue behind cutting edge 73 and the blade 70A is drawn along the desired path 8 by driver 90 such that any pressure that could force the cutting edge of the blade from the guiding edge of the reference member is alleviated/.

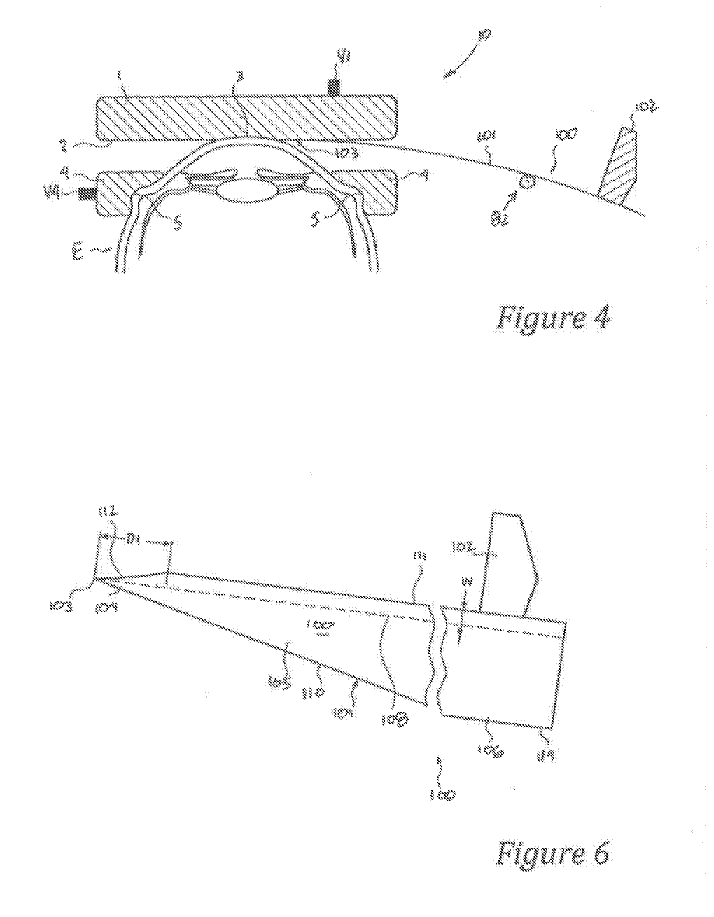

FIG. 4 illustrates yet another embodiment of the surgical apparatus 10 including the above-described RRM elements as well as a flexible blade 100 and a manipulating block 102. The manipulating block 102 can be mounted in a specified arrangement, e.g., via fulcrum 82, for applying desired forces and movements to the plate member 101 of the blade 100 in a predetermined manner. In particular, the blade 100 is mounted to the surgical apparatus such that the plate member 101 is bent or flexed in a predetermined manner, while contacting the guiding surface of the reference member 1 which comprises the surface forming the guiding surface 2. For this reason, a predetermined bending moment is applied. Moreover, a movement of the blade 100, in particular, of the manipulating block 102 can be induced such that the blade 100 can be moved in a direction substantially aligned to guiding surface. An additional arrangement for applying an oscillating movement to the blade 100, e.g., to the manipulating block 102, in the orthogonal direction can be provided to provide a cleaner cut.

FIG. 4 shows the surgical apparatus in a situation in which the main tissue section E is held by the fixing arrangement 4, wherein the tissue portion e to be removed in the cutting process is placed in the cavity 3. In preparation of the cutting process, the blade 100 is mounted in the surgical apparatus in the way discussed above. Prior to the performance of the operation, the relationship of the surface of the guiding surface 2 and the blade 100 is as shown in FIG. 4. In particular, due to a bending moment applied by the manipulating member 102 to the plate member 101 about Z, the plate member 101 is flexed to a predetermined extent.

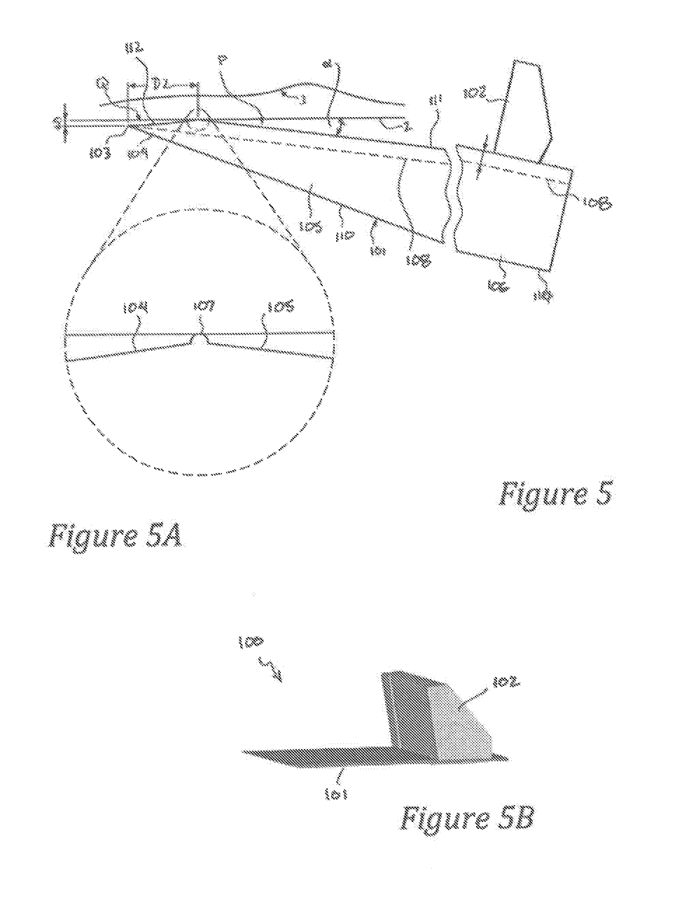

In this situation, which is shown in FIG. 5, the cutting edge 103 is spaced from the surface forming the guiding surface 2 by a cutting edge distance S. Moreover, the contact ridge 107 is in sliding contact to the surface forming the guiding surface 2 with a predetermined pressure created as counterforce in the direction of arrow Y due to the bending or flexing of the plate member 101. A space P as rear space is formed in the area between the guiding surface and the first main surface in of the blade 100 which space is located opposite to the cutting edge 103 in relation to the contact ridge. As shown in FIG. 5, the biasing mechanism maintains the blade (100) at an angle (α) between 1° and 20° relative to a surface of the reference member or, preferably between 1° and 20°, or 2° and 10°, or 2° and 6° relative to the reference member surface. In the situation shown in FIG. 4, the operation can be initiated.

In order to perform the intended cutting operation, which includes a cutting or slicing action to at least partially remove the tissue portion e from the main tissue section E, the blade 100 is moved from the position shown in FIG. 4 towards the tissue portion e. In order to achieve this, the blade is moved by applying a predetermined movement to the manipulating member 102 along the surface forming the guiding surface 2. In specific cases, the surface can be plane or straight as shown in FIG. 4. As indicated above, it is also possible to provide curved or otherwise shaped surfaces, which form a guiding edge 2.

Optionally, oscillating movement along the direction orthogonal to the direction of blade movement can be applied to the manipulating member 102. For promoting the cutting process, the blade 100 is moved towards the tissue portion e and driven into the tissue with the cutting edge 103. The cutting edge 103 separates the tissue portion e at least partially from the main tissue section E. The optional oscillating movement along the direction of arrow Z enhances the cutting process due to the fact that the cutting edge is oscillated in a direction that is substantially aligned to the direction in which the cutting edge 103 extends.

While the blade 100 is moved into the tissue, the bending moment applied to the plate member 101 by the manipulating member 102 is maintained such that the contact ridge 107 or the blade 100 is kept in pressure contact in the direction of arrow Y to the surface forming the guiding surface 2. In the course of moving the cutting edge 103 into the tissue, an expansion of the tissue portion e can occur such that the volume of the tissue portion e can become greater than the volume of the cavity 3 in which the tissue portion e is held. The space P shown in FIG. 5, which is provided in the arrangement according to the present invention can accommodate such expansion such that fiction, pressure-induced deviation of the cutting edge, or any other disturbance in the course of the cutting process can be avoided. On the other hand, a space Q forming a front space between the first taper surface 112 and the surface of the guiding surface 2 is very small due to the short distances D2 and S as discussed above.

Returning to FIG. 4, the movement of the blade 100 into the tissue is continued until the tissue portion e is separated from the main tissue section E to the desired extent. In a specific application, the tissue portion e is to be separated from the main tissue section E completely such that the movement of the blade 100 is continued to an extent that the cutting edge 103 completely sweeps the cavity 3 and completely removes the tissue portion e from the main tissue section E.

In this situation, the tissue portion e can be maintained in the cavity 3 due to the applied reduced pressure and/or adhesive as explained above. In this situation, the cutting or slicing process is completed and the blade 100 can be retracted. As alternative, the fixing arrangement 4, e.g. the suction ring, can be controlled in position in relation to the reference member such that the main tissue section E is spaced from the separated tissue portion e.

According to the concept of the present invention, the blade 100 is designed with a specific arrangement, which allows a space P being provided for accommodating an expansion of tissue in the course of the cutting process. Moreover, the specific inventive geometry of the blade increases the accuracy of the position of the cutting edge with respect to the intended cutting surface even with deviating operational conditions. In the inventive surgical apparatus, only a single surface is required for guiding the blade with a satisfactory accuracy. As such, the invention relates to the specific design of the blade 100 as discussed above, while the surgical apparatus contributes to the inventive solution stated above.

The blade element 100 of the present invention will be explained further with reference to FIG. 6, which illustrates an example of the blade 100 in a schematic view. (The terms “blade,” “blade member” and “blade element” are used interchangeably throughout this description.) The blade 100 is formed by a plate shaped plate member 101, which is in the present embodiment formed as flat rectangular member. The rectangular member can be manufactured by a single piece of metal sheet, e.g. spring steel or the like, providing a specific flexibility. In particular, the plate member 101 can be bent due to its elasticity such that the longitudinal dimension of the plate member 101 follows a bending line.

Again with reference to FIG. 6, at a first longitudinal end portion of the plate member 101, a tip section 104, 105 is provided. The tip section 104, 105 extends from a main section 106 of the plate member 101. The main section 106 of the plate member 101 covers a major longitudinal portion of the plate member 101. As shown in the drawings, the tip section includes a front tip section 104 and a rear tip section 105. The first longitudinal end portion of the plate member 101 is also provided with a cutting edge 103 which is formed by a sharpened tip end which serves as cutter used in the cutting process. The sharpened tip end can be machined in a known manner.

A manipulating member 102 can mounted at a second longitudinal end portion of the plate member 101. The manipulating member 102 has a predetermined shape such as a box shape and is mounted to one of two opposite sides of the plate member 101 in the shown example. The manipulating member 102 can be mounted to the plate member 101 by gluing, welding, riveting, screwing or any other method. The purpose of the manipulating member 102 is to introduce a force to the plate member 101, in particular, a bending moment for bending or flexing the plate member 101 and a force for moving the blade 100 in the surgical apparatus. The manipulating member 102 can be provided with recesses or protrusions for transmitting a force for bending or moving the plate member 101 from a driver (not shown).

In the tip section 104, taper surfaces 110, 112 are provided on both sides of the plate member 101 as explained below. The tip section 104 is formed by a first taper surface 112 which is inclined with respect to a first main surface in formed in one side of the plate member 101. In particular, the first taper surface 112 can be formed in the side of the plate member 101 at which the manipulating member 102 is arranged. The first taper surface 112 is inclined with a predetermined angle in relation to the first main surface 111. It is noted that the offset of the main section 106 of the blade 100 created by the bent or flexed condition is not reproduced for sake of clarity.

On the other side opposite to the side where the manipulating member 102 is arranged, a second taper surface 110 is provided. The above mentioned first taper surface 112 and the second taper surface 110 merge in the tip end of the plate member 101 for forming the above discussed cutting edge 103.

With further reference to FIG. 6, the location of a transition of the first taper surface 112 and the first main surface in of the plate member 101 is spaced at a cutting edge distance Di from the cutting edge 103 in the longitudinal direction of the plate member 101. In this context, the longitudinal direction is defined as the direction aligned to the symmetry axis of the main section 106 of the plate member 101. In this context, in a preferred embodiment, the distance between the first main surface in and a second main surface 114 forming the main section 106 of the plate member 101 is substantially constant throughout the main section 106. As such, the surfaces in and 114 are parallel in relation to each other. As already stated, it is not excluded that the main section 106 comprises deviating parts such as holes and protrusions as long as the majority of the main section 106 fulfils this requirement. For a correct explanation, the distance between the cutting edge 103 and the contact ridge 107 is shown as cutting edge distance (mounted) D2 in FIG. 4 using as basis the longitudinal direction of the movement of the blade 100.

As can be derived from FIG. 6, the blade 100 is shown in a relaxed state in which the first main surface 111 is substantially flat or plane and the offset plane distance W between the first main surface in and a virtual offset plane 108 which is oriented parallel to the first main surface in of the main section 106 is specifically offset as discussed below.

In particular, the above mentioned offset plane 108 which is oriented parallel to the first main surface 111 is virtually arranged such that this offset plane 108 meets or includes the cutting edge 103. Due to the specific arrangement of the first taper surface 112 and the second taper surface 110, the position of the offset plane 108 is offset from a symmetry plane or symmetry axis of the main section 106 of the plate member 101. In particular, this offset plane 108 is offset towards the first main surface in such that the offset plane distance W is less than 50 percent of the thickness of the plate member 101 in the main section 106. In the shown embodiment, the distance W is approximately 15 percent of the thickness of the main section 106 of the plate member 101. The extent of offset arrangement can be set as required as long as the distance W is less than 50%, preferably less than 40%.

A contact ridge 107 is preferably formed at the transition of the first taper surface 112 and the first main surface 111, as best shown in FIG. 5A. The protrusion is formed at the transition between the first taper surface 112 and the first main surface 111. In the present embodiment, the protrusion is formed with a circular shape and extends from both the first taper surface 112 and the first main surface in. Preferably, the contact ridge 107 is formed so as to extend throughout the lateral area of the plate member 101. While the embodiment shows the contact ridge 107 with a specific shape, the invention is not restricted to this shape. Rather, any shape of the contact ridge 107 can be selected as long as it is located in the transition of the first taper surface 112 and the first main surface in. Moreover, it is possible to provide the contact ridge 107 as step formed merely by the transition of the first taper surface 112 and the first main surface in without adding any material of providing any specific shape. Therefore, the contact ridge 107 can also be a line-shapes step created by the different angles of the first taper surface 112 and the first main surface 111 at the transition thereof.

Returning to FIG. 5, the contact ridge 107 can be provided for contacting a guide in particular a guiding surface 2 of a reference member formed in the surgical apparatus. The guiding surface 2 is shown in FIG. 5, which is in contact with the contact ridge 107 while the plate member 101 is flexed or bent forming a bending line along the longitudinal direction of the plate member 101. In the situation shown in FIG. 5, a force resulting from the plate member 101 being flexed or bent acts between the contact ridge 107 and the guiding surface 2 formed in the surgical apparatus. The force acts for avoiding a departure of the plate member 101 from the guiding surface 2 such that guidance is provided by the above-mentioned force. At the same time, a contact between the guiding surface 2 and the cutting edge 103 is avoided due to the provision of the first taper surface 112 in the tip section of the plate member 101. Moreover, the arrangement is such that a space P between the blade 100 and the guiding surface 2 can be provided in the area located opposite with respect to the contact ridge 107 from the cutting edge 103. Consequently, areas other than the contact ridge 107 are not in contact with the guiding surface 2.

As consequence, the cutting edge 103 is spaced from the guiding surface 2 as shown in FIG. 5 by a cutting edge distance S. According to the inventive arrangement of the blade 100, the blade 100 is arranged for providing a guidance function by a force exerted from the contact ridge 107, which results from the plate member 101 of the blade 100 being flexed by applying a bending moment to the manipulating block 102. Due to the specific arrangement of the cutting edge 103 with respect to the symmetry plane or symmetry axis of the main section 106 of the plate member 101, a deviation of the cutting edge distance S due to differing or deviating flexing states of the plate member 101 are reduced.

This surprising advantage is achieved due to the fact that a neutral axis of the main section 106 of the plate member 101 does not correspond to the offset plane 108 which is parallel to the first main surface in and which includes the cutting edge 103. In other words, the offset plane 108 which is parallel to the first main surface in and which includes the cutting edge 103 is offset from the neutral axis of the main section 106 towards the side at which the contact ridge is provided in order to reduce the influence of deviating flexing states of the plate member 101 to the cutting edge distance S between the cutting edge 103 and the guiding surface 2 provided in the surgical apparatus. This effect is achieved by reducing a bending or flexing deviation at the tip section including the cutting edge 103 such that a deviation of the bending line of the plate member 101 has a minimum influence on the bending of the tip section in the range between the contact ridge 107 and the cutting edge 103.

In the illustrated embodiment, the offset plane distance W is optimally less than 40% of the thickness of the plate member 101 in the main section 106. Even better results are achieved with an offset plane distance W of less than 30% of the thickness of the main section 106, wherein the shown embodiment discloses an offset plane distance W of approximately 15% of the thickness of the main section 106.

The cutting edge distance D1 in the embodiment (shown in FIG. 6) can be set to a range of 5 to 50 micrometers. The cutting edge distance D1 is preferably 20 to 40 micrometers in order to reach a satisfactory accuracy. The cutting edge distance S (shown in FIG. 5) between the cutting edge 103 and the guiding surface 2 can preferably be in the range of 0.5 and 20 micrometers, more preferably in some applications between 2 and 10 micrometers. The thickness of the main section 106 of the plate member 101 is preferably set to 25 to 250 micrometers. Better results are achievable by setting the thickness of the main section 106 to 50 to 100 micrometers. This arrangement is in particular of advantage in case that spring steel is used as material for forming the plate member 101.

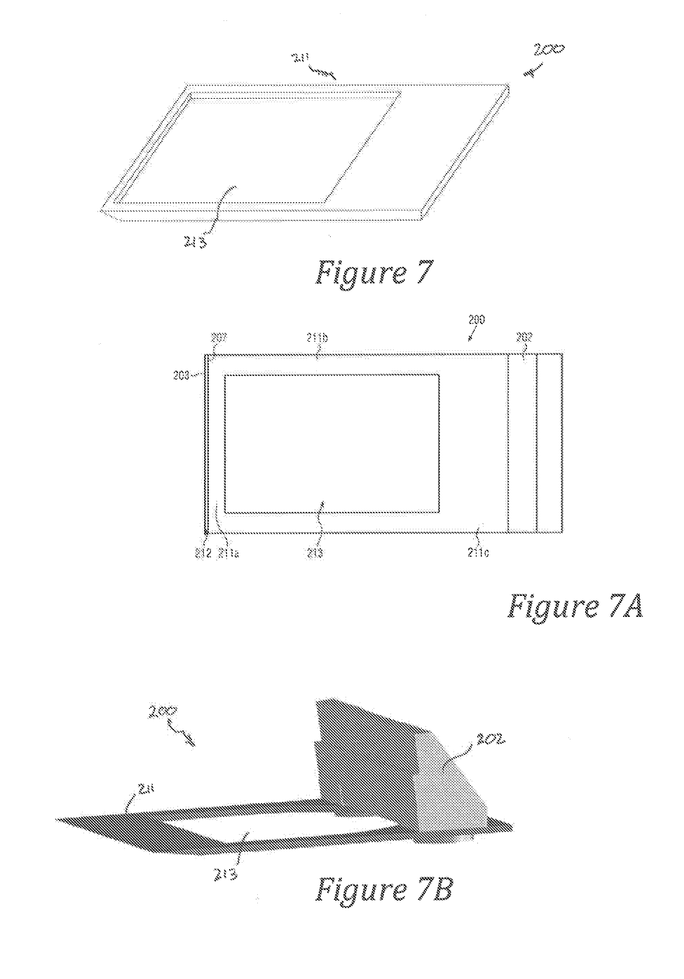

While the above-explained embodiment relates to a specific shape of both the blade and the surgical apparatus, it is possible to deviate from the above explained arrangement without departing from the concept of the present invention. In particular, the shape of the blade element can be modified as e.g. shown in FIGS. 7A and 7B. While the main features of the modified blade element 200 are the same as explained above, differences in relation to the above explained blade element 100 are specified below.

The blade element 200 shown in FIG. 7A-7B comprises a cutting edge 203 at one end thereof. In an end portion opposite to the cutting edge 203, a manipulating block 202 is provided. A contact ridge 207 is arranged in the blade element 200 in close relationship to the cutting edge 203 as in the blade element 100. The plate member according to this modification is similar as in the previous explanation except the provision of window 213 which is provided in the main section of the blade element 200. In particular, the plate member is formed by a front continuous section 211a in the area of the cutting edge 203. To this front continuous section 211a, a portion having the window 213 is connected, wherein the window 213 is defined by two leg elements 211b. In the end portion where the manipulating block 202 is arranged, a rear continuous section 211C is arranged.

The above modification provides alternative properties in relation to the flexibility of the plate member which can advantageously employed in the surgical apparatus. Moreover, the window 213 provides an increase of the space P discussed above such that any disturbance between tissue expanding in the process of cutting can be avoided. Moreover, the visibility of elements to be cut is improved due to the fact that the window 213 allows the user of the apparatus to view e.g. the tissue in the cutting process. The remaining advantages and structures are the same as in the above-explained embodiments.

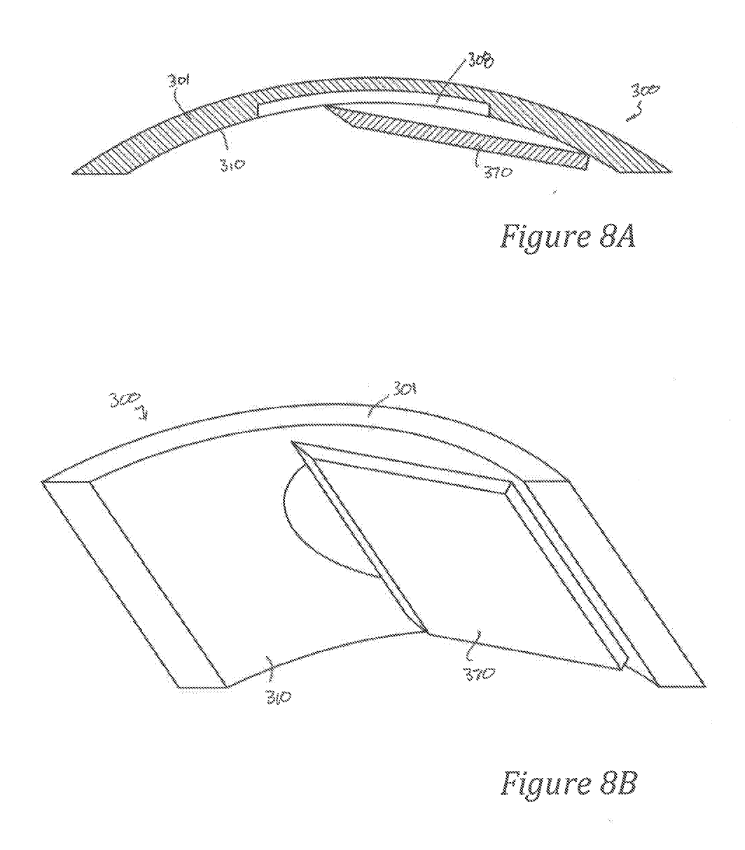

In FIGS. 8A and 8B another embodiment 300 is presented, in which the blade 370 is flat, but the reference member 301 has a curved surface 310. The curvature in FIG. 8A is two dimensional only as can be seen in the perspective view (FIG. 8B). This design works in all aspects analogous to the previously described embodiments. (The shape of the reference member's cavity can be modified to achieve the desire shape of the resected tissue section.) The curvature of guide surface 310 can be for example a part of a circle or a part of an ellipse.

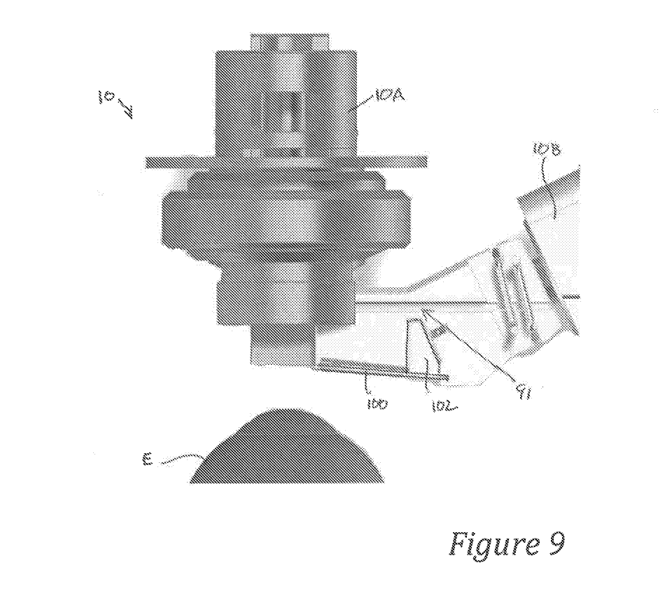

FIG. 9 is a schematic, partially cut-away, side view of an ocular surgery apparatus 10 according to the invention having a stationary (rigid reference member) component 10A for holding an eye E and a translatory assembly 10B that holds blade 100. Translatory motion is enabled by rail slots 91, which engage rails (not shown) within the stationary component 10A.

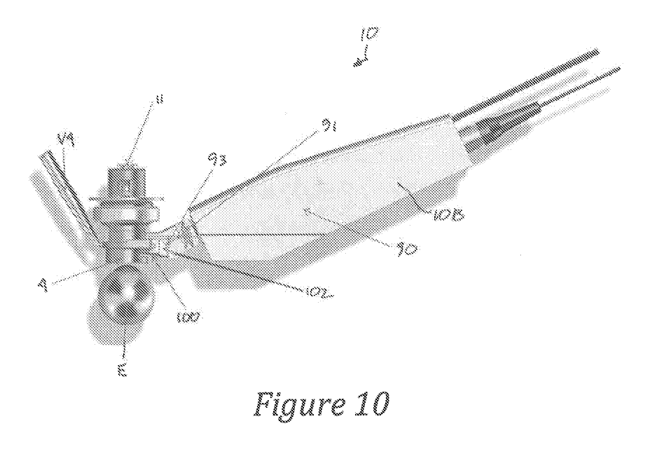

FIG. 10 is a more detailed schematic side view of the apparatus 10 of FIG. 9 also showing an associated eye-fixation suction ring 4 and vacuum port V4 coupled to the stationary RRM component 10A. Translatory assembly 10B is partially cut-away to illustrate the blade 100, the blade manipulating block or handle 102, as well as oscillatory pin 93 and a rail slot 91, which form part of the driver 90. The driver 90 provides for oscillatory (in and out of the plane of the figure) and translational (right to left) movements of the blade 100.

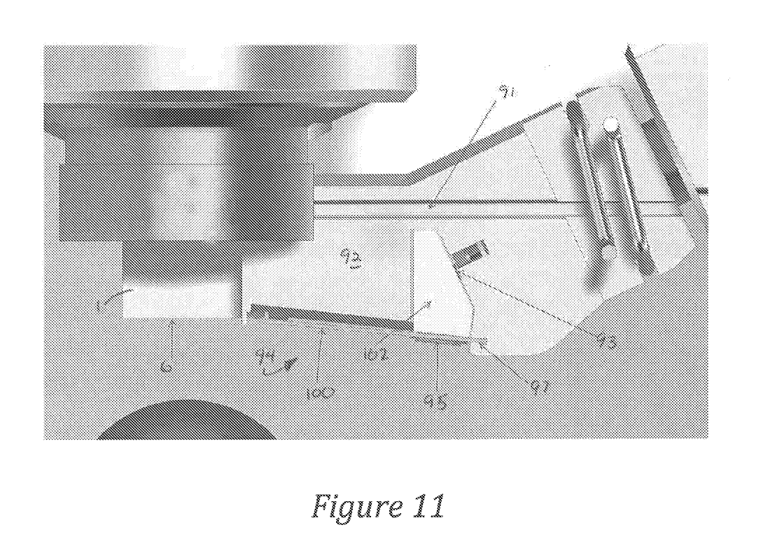

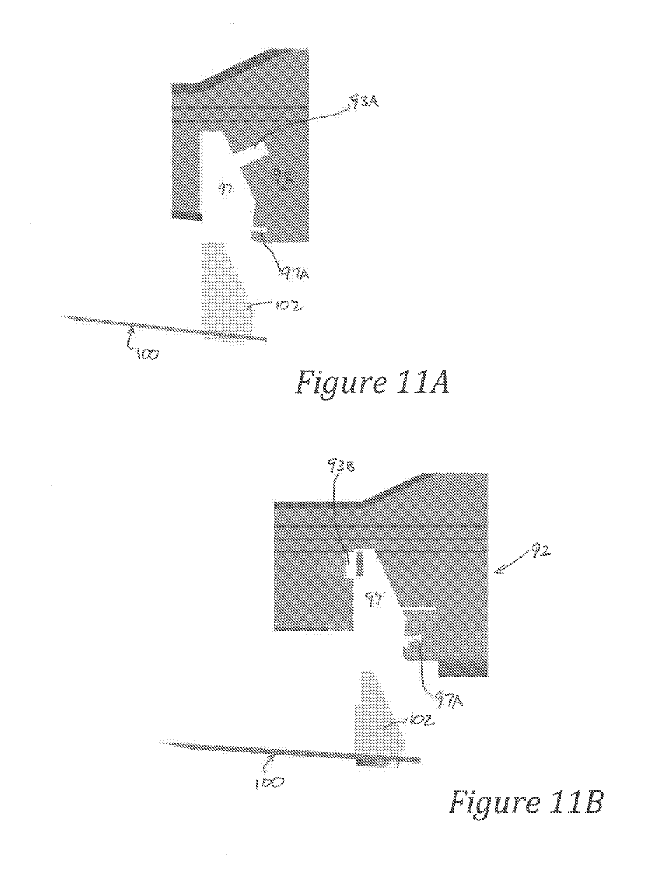

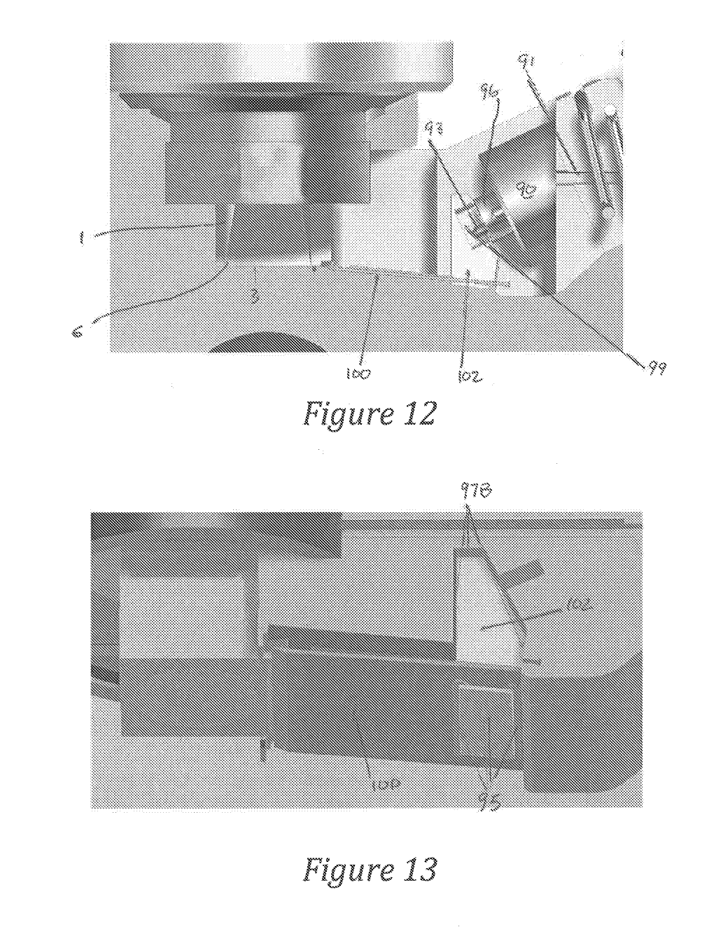

FIG. 11 is a partially cut-away, side view of the apparatus of FIGS. 9 and 10 with the suction ring and drive mechanism removed to further illustrate the rail slot 91 for translational movement and the attachment of the blade to drive head 92. The blade 100 is joined to a manipulating block 102 by fastener 95. The blade and block assembly 94 is removably coupled to drive head 92 via recess 97 and slot 97A which restrains the blade's degrees of freedom. In the illustrated embodiment, the block 102 is non-rectangular in shape (e.g., a four or more-sided polygon) and can only be slid into the driver head 92 sidewise such that its irregular polygonal shape prevents dislodgement except by reverse sideways extraction. Once the blade and block assembly 94 is inserted into the slot 97, oscillatory pin 93 can be engaged within a slot (shown in FIG. 12) in the block 102 to further constrain movement of the blade 100. In the illustrated embodiment, the drive head 92 and rail slot 91 act as the bearing that applies a biasing force to the blade such that it remains in contact with the guiding edge 6 of the rigid reference member 1.

FIGS. 11A and 11B illustrate two alternative mechanisms for joining blade assembly 100 to the translatory drive head 92. In FIG. 11A the mounting block 102 is inserted into recess 97 and locked in place by oscillatory pin (not shown) in pinhole 93A. In FIG. 11B mounting block 102 is likewise inserted into recess 97 and locked in place by snap-fitting 93B.

FIG. 12 is another partially cut-away, side view of the apparatus of FIGS. 9-11, showing a mechanism for imparting oscillatory motion to the blade of the apparatus. Eccentric pin 93 of driver 90 engages slot 99 of the blade 102. Rotation of shaft 96 caused pin 93 to move in a circular manner within slot 99, thus causing the block 102 and the blade 100 to oscillate in a direction generally orthogonal to the translatory movement of the blade and block assembly 94 along the guiding edge 6 of the reference member 1. (Because this figure is generally drawn to scale, the curvature of the cavity 3 is largely imperceptible.)

FIG. 13 is a schematic, partially cut-away, perspective view of the apparatus of FIGS. 9-12 showing the assembly 94 (comprising blade 100 and block 102) inserted into a mating receptor or keyhole 97 within a driver element 90 of the apparatus. Slot walls 97B retrain the blade's mounting block's movement. In this illustration, the blade 100 and block 102 are joined together by fastener 95. The apparatus is positioned to allow blade 100 to be drawn across the guiding surface 2 of the rigid reference member 1 to sever a tissue portion (not shown) within cavity 3 from a main section of tissue (not shown). The tissue to be resected can be held with the cavity 3 by either suction or glue applied to the cavity surface prior to engagement.

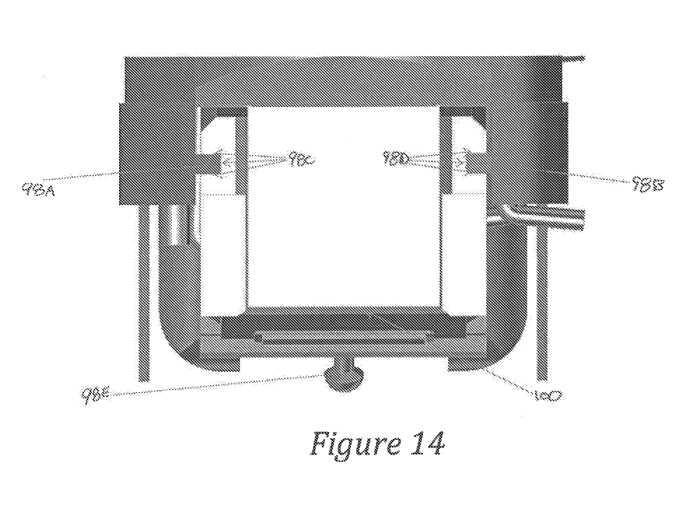

FIG. 14 is a cut-away, front view of the stationary element of the apparatus of FIGS. 9-13 showing rails for engaging the translatory blade and drive elements of the apparatus. This front view shows how the translatory elements of the apparatus accomplish tissue resection. Left and right rails (98A and 98B) within the tissue fixation base 96 are engaged by the walls 98C and 98D of complementary rail slots 91. Docking pin 98E can provide a connection to driver 90 (not shown) for implementing translator motion of the blade assembly. Various mechanisms, can be incorporated into drive 90 (not shown) to effect movement of blade 100 into and out of the plane of the drawing. For example driver 90 can comprise a linear motor. Alternatively, translational movement can be controlled manually.

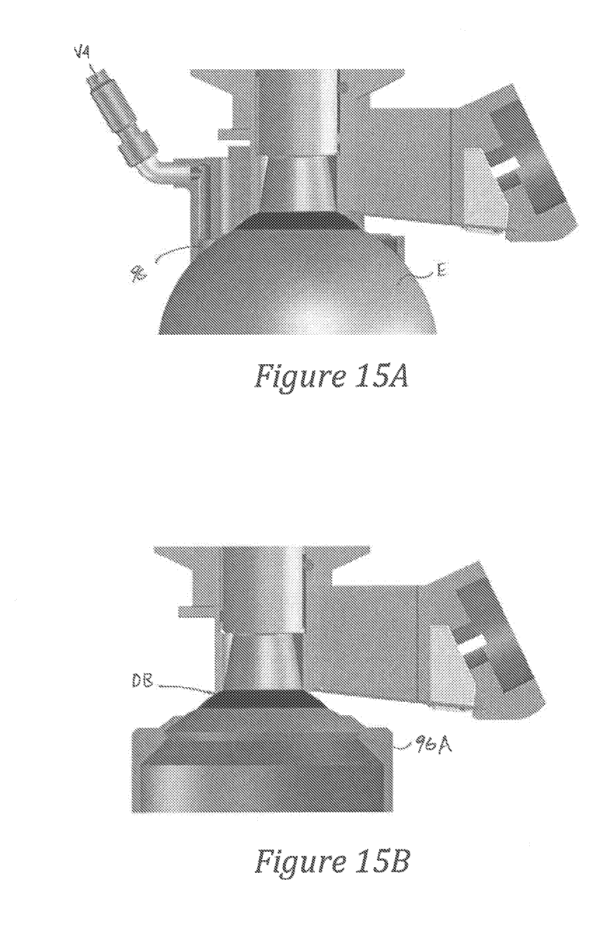

FIG. 15A is a schematic, partially cut-away, side view of an eye fixation ring according to one embodiment of the invention. Ring 96 can be configured to surround and engage the sclera of eye E. Fixation is maintains by the application of negative pressure via vacuum port V4. In FIG. 15B another embodiment of the invention is shown utilizing a tissue fixation stage 96A in lieu of the eye fixation device of FIG. 15A. The embodiment of FIG. 15B can be useful, for example, in sculpting donor tissue, such as a corneal donor button DB prior to transplantation.

While the above explanation has been provided based on the application of the surgical apparatus to an eye surgery, the surgical apparatus as well as the blade element are applicable to a general surgical cutting or slicing process of any type of tissue. In particular, the apparatus can be applied to those types of surgical slicing or cutting processes in which a specific high accuracy is required.

### REFERENCE NUMERALS

[This Section Will be Deleted from Final Application]
1 REFERENCE MEMBER
2 GUIDING EDGE OR SURFACE
3 CAVITY
4 FIXING ARRANGEMENT (SUCTION RING)
5 FIXING RECESS
6 GUIDING SURFACE
8 DESIRED PATH
9 DEVIATION FROM DESIRED PATH
10 SURGICAL APPARATUS
10A STATIONARY COMPONENT OF APPARATUS 10 (FIG. 9)
10B TRANSLATORY COMPONENT OF APPARATUS 10 (FIG. 10)
70 BLADE ELEMENT
70A BLADE (FIG. 3)
72 CURVED SEGMENT OF BLADE (FIG. 3)
73 CUTING EDGE
80 BIASING MECHANISM (BEARING)
82 FULCRUM
84 BLADE SHANK (CURVED)
90 MOTOR/DRIVER
91 RAIL SLOT
92 DRIVE HEAD
93 OSCILLATORY PIN
94 BLADE AND BLOCK ASSEMBLY
95 FASTENER
96 STATIONARY TISSUE FIXATION BASE
96A TISSUE FIXATION STAGE
97 RECESS (KEYHOLE) CONNECTING BLOCK TO DRIVE HEAD
97A SLOT PORTION OF RECESS 93
98A/98B RAILS
98C/98D RAIL SLOT WALLS
98E DOCKING PIN
99 SLOT FOR ECCENTRIC PIN
100 BLADE
101 PLATE MEMBER
102 MANIPULATING MEMBER/HANDLE/BLOCK
103 CUTTING EDGE
104 FRONT TIP SECTION
105 REAR TIP SECTION
106 MAIN SECTION
107 CONTACT RIDGE
108 OFFSET PLANE
110 SECOND TAPER SURFACE
111 FIRST MAIN SURFACE
112 FIRST TAPER SURFACE
114 SECOND MAIN SURFACE
200 BLADE ELEMENT
202 MANIPULATING BLOCK
203 CUTTING EDGE
207 CONTACT RIDGE
211a FRONT CONTINUOUS SECTION
211b LEG ELEMENT
211c REAR CONTINUOUS SECTION
212 MANIPULATING BLOCK
213 WINDOW
300 CURVED EMBODIMENT
301 CURVED REFERENCE MEMBER
310 CURVED GUIDE SURFACE
370 BLADE ELEMENT (FIG. 8)
D1 CUTTING EDGE DISTANCE
D2 CUTTING EDGE DISTANCE (MOUNTED)
DB CORNEAL DONOR BUTTON
e TISSUE PORTION TO BE RESECTED
E MAIN TISSUE SECTION
P REAR SPACE
Q FRONT SPACE
S CUTTING EDGE DISTANCE
W OFFSET PLANE DISTANCE
V1 VACUUM PORT
V4 VACUUM PORT

1. Surgical apparatus for performing a tissue cutting process to cleave a tissue portion from a main tissue section comprising:
a reference member having a cavity for holding a tissue portion subject to the tissue cutting process and a blade guiding surface,
a blade having a cutting edge for cutting the tissue portion to at least partially sever it from the main tissue section, the blade adapted for slidable engagement with the reference member, such that the blade moves along the guiding surface of the reference member to perform the cutting process, and
further characterized by a biasing mechanism for moving the blade along the guide surface such that a pressure-relieving space is formed between the blade and the tissue section (e) upon cleavage to avoid deviation of the cutting edge from a desired path.

2. The apparatus of claim 1 wherein the biasing mechanism maintains the blade at an angle (α) between 1° and 20° relative to a surface of the reference member or, preferably, between 1° and 20°, or 2° and 10°, or 2° and 6° relative to the reference member surface.

3. The apparatus of claim 1 wherein the biasing mechanism further comprises a bearing, which maintains the blade at an incline relative to a surface of the reference member as the blade is moved along the guide element to create the space between the blade and the tissue section (e) upon cleavage.

4. The apparatus of claim 1 wherein the biasing mechanism further comprises a bent surface on the blade such that the space is formed by the curve of the blade as the blade is moved along the guide element to create the space between the blade and the tissue section upon cleavage.

5. The apparatus of claim 1 wherein the biasing mechanism further comprises a torque mechanism whereby the blade element is flexed by applying a bending moment to the blade element and, optionally, wherein the applied torque at a distal end of the blade is between 10 and 200 grams, or between 30 and 70 grams.

6. The apparatus of claim 1 wherein the blade further comprises a transverse guide-engaging ridge substantially parallel to the blade edge, which is the contact surface for the slidable engagement of the blade with the guide element.

7. The apparatus according to claim 6 wherein the blade is mounted in the surgical apparatus in a flexed state such that the guide-engaging ridge is forced against the guiding surface for defining the movement path in the cutting process.

8. Surgical apparatus according to claim 1, further comprising a driver for driving the blade along the guiding surface in a cutting direction such that the cutting edge leaves the tissue portion and the main tissue section.

9. Surgical apparatus according to claim 8, wherein the driver further comprises an oscillator for transmitting an oscillating movement to the blade, the oscillating movement being directed in a substantial lateral direction in relation to the cutting direction.

10. Surgical apparatus according to claim 1, wherein the cavity has an opening on the same side as the guiding surface and the cavity is arranged for accommodating and/or holding the tissue portion to be removed from the tissue main section in the cutting process, and wherein the reference member further comprises an source of negative pressure for applying reduced pressure to the cavity for holding the tissue portion to be removed in the cutting process and, optionally, wherein at least a portion of the reference member defining the cavity is formed as air permeable structure for applying reduced pressure to the cavity.

11. Surgical apparatus according to claim 1, wherein a surface of the blade facing to the guiding surface is inclined with respect to the guiding surface in the cutting process such that a space is formed between the blade element and a cut surface of the tissue portion to be removed in the cutting process.

12. Surgical apparatus according to claim 1, further comprising a fixing arrangement for at least partially fixing the main tissue section (E) in a predetermined relationship to the reference member.

13. Surgical apparatus according to claim 12, wherein the fixing arrangement comprises a fixing recess for applying a low pressure to a portion of the main tissue section for fixing the main tissue section in relation to the fixing arrangement and/or the reference member.

14. Surgical apparatus according to claim 1, wherein the main tissue section is an eye and the tissue portion to be removed in the cutting process is a portion of a cornea of the eye.

15. A blade for use in a surgical apparatus for cutting tissue along a predetermined path, the blade comprising:
a shank plate having first and second opposite sides,
a cutting edge formed at a first longitudinal end portion of shank,
a contact ridge formed on a first surface of shank plate at a predetermined distance from the cutting edge with respect to a longitudinal direction of the shank plate,
wherein the contact ridge is arranged for slidable engagement with a guiding surface formed in the surgical apparatus.

16. The blade of claim 15 wherein the contact ridge of the blade is further adapted to be held in slidable engagement by a biasing mechanism.

17. The blade of claim 15 wherein the Blade is flexible and the biasing mechanism further comprises a manipulating block is adapted to apply a bending moment force to the blade to induce flexure.

18. The blade according to claim 15, wherein the shank plate further comprises:
a main section having a first main surface at the first side of the plate and a second main surface at the second side of the plate and
a tip section extending from the main section and including the cutting edge.

19. The blade according to claim 18, wherein the main section is formed with a substantially constant thickness between the first main surface and the second main surface.

20. The blade according to claim 15, wherein the tip section is formed by a first taper surface on the first side of the plate and a second taper surface at the second side of the plate, the first and second taper surfaces merging into the cutting edge.

21. The blade according to claim 20, wherein the position of the cutting edge is offset towards the first side of the plate with respect to a symmetry plane defined in the main section between the first and second main surfaces.

22. The blade according to claim 18, wherein a distance of a plane which is oriented parallel to the first main surface of the main section and which includes the cutting edge is less than 40%, preferably less than 30% of the thickness of the main section.

23. The blade according to claim 20, wherein the contact ridge is formed at a transition of the first taper surface and the first main surface.

24. The blade according to claim 23, wherein the contact ridge protrudes from a first taper surface and a first main surface.

25. The blade according to claim 15, wherein a distance between the contact ridge and the cutting edge in the longitudinal direction of the plate member is 5 to 50 micrometers, preferably 20 to 40 micrometers.

26. The blade according to claim 15, wherein a thickness of the main section is 25 to 250 micrometers, preferably 50 to 100 micrometers.

27. The blade according to claim 15, wherein a slidable engagement between a guide surface and the contact ridge is achieved based on a force, which acts between the guiding surface and the contact ridge.

28. The blade according to claim 15, wherein the contact ridge is linear or curved in shape.

29. The blade according to claim 15, wherein the contact ridge is a continuous structure or composed of a plurality of ridge segments.

30. The blade according to claim 17, wherein the manipulating block has an irregular shape, optionally a polygonal shape to fit into a locking polygonal keyhole, to prevent dislodgement of the blade or cutter during use.
